(19) United States
(12) Patent Application Publication
(10) Pub. No.: US 2011/0251195 A1
(43) Pub. Date: Oct. 13, 2011

(54) ADRB2 GENE POLYMORPHISM ASSOCIATED WITH INTRAOCULAR PRESSURE RESPONSE TO TOPICAL BETA-BLOCKERS

(75) Inventors: Catherine Anne McCarty, Marshfield, WI (US); James K. Burmester, Marshfield, WI (US); Bickol N. Mukesh, Mansfield, TX (US); Richard B. Patchett, Marshfield, WI (US); Russell Alan Wilke, Brookfield, WI (US)

(73) Assignee: Marshfield Clinic, Marshfield, WI (US)

(21) Appl. No.: 13/166,845

(22) Filed: Jun. 23, 2011

Related U.S. Application Data
(62) Division of application No. 12/474,441, filed on May 29, 2009.
(60) Provisional application No. 61/057,047, filed on May 29, 2008.

(51) Int. Cl. A61K 31/5377; C12Q 1/68; A61K 31/4704; A61P 27/06; A61K 31/138; A61K 31/222

(52) U.S. Cl. 514/236.2; 514/652; 514/546; 514/312; 435/6.11

(57) ABSTRACT

The invention provides a single nucleotide polymorphism (SNP) rs1042714 in the human ADRB2 gene (Gln27Glu) associated with a clinically meaningful reduction in intraocular pressure (IOP) in a human following treatment with a topical beta-blocker. Nucleic acids comprising the SNP are used to screen glaucoma-afflicted individuals to thereby provide an improved method for genotype-based prescribing of beta-blockers in glaucoma management.

### CROSS REFERENCE TO RELATED APPLICATION

The present application is a divisional of U.S. patent application Ser. No. 12/474,441, filed May 29, 2009, which claims the benefit of U.S. Provisional application 61/057,047, filed May 29, 2008, both of which are incorporated herein by reference in their entirety for all purposes.

### STATEMENT REGARDING FEDERALLY SPONSORED RESEARCH

Not applicable.

### FIELD OF THE INVENTION

The present invention is related to management of glaucoma. More particularly, the invention is directed to an ADRB2 gene single nucleotide polymorphism associated with a clinically meaningful reduction in intraocular pressure response in a human following treatment with a topical beta-blocker.

### BACKGROUND OF THE INVENTION

Glaucoma is a progressive optic neuropathy with an estimated prevalence of 1.86% in the United States population over the age of forty. Previous research has shown that half of glaucoma cases in the community are undiagnosed. Timely diagnosis and treatment of glaucoma are necessary to prevent irreversible degeneration of retinal ganglion cells and concomitant vision loss. The goal of glaucoma management is to lower intraocular pressure (“IOP”) as lowering IOP has been shown in the clinical setting to reduce the risk of visual loss. Treatment options include, e.g., pharmacologic agents, laser treatment and surgery.

Genetic variability, along with compliance, environment and eye/systemic disease is thought to contribute to the overall IOP response to glaucoma medications. Cytochrome P450 (CYP) 2D6 metabolizes the beta-blocker timolol, in vivo, and CYP2D6 gene polymorphisms have been shown to be associated with timolol-related outcomes. Topical beta-blockers such as timolol are currently the least expensive class of agents used to lower IOP. Topical beta-blockers have been shown to have systemic effects. Outcomes related to systemic absorption of timolol are apparently related to CYP2D6 genotype, as well as antidepressants and other drugs known to modulate the activity of CYP2D6.

In order to provide improved methods of glaucoma management, it is highly desirable to understand the relationship between genetic variability and IOP response to glaucoma medications. Therefore, a need exists for biomarkers identified as associated with clinically meaningful reductions in IOP following treatment with topical beta-blockers.

### SUMMARY OF THE INVENTION

The present invention is directed to an ADRB2 gene single nucleotide polymorphism associated with a clinically meaningful reduction in intraocular pressure response following treatment with a topical beta-blocker. The invention is based on the inventors' recent efforts to determine if candidate pharmacodynamic and pharmacokinetic gene polymorphisms are associated with IOP response to topical beta-blockers.

Accordingly, the invention provides in a first aspect a method for identifying a glaucoma-afflicted human who will experience a clinically meaningful reduction in intraocular pressure upon treatment with a topical beta-blocker. Such a method includes steps of detecting a CC genotype at coding single nucleotide polymorphism (SNP) rs1042714 in the ADRB2 gene (Gln27Glu) in a nucleic acid sample from the human, wherein the presence of the CC genotype indicates that the human will experience a clinically meaningful reduction in intraocular pressure upon treatment with a topical beta-blocker as compared to a human lacking the CC genotype and undergoing the same treatment.

In a preferred embodiment, the human identified as having the CC genotype exhibits a greater than twenty percent decrease in intraocular pressure upon treatment with a topical beta-blocker as compared to a human undergoing the same treatment but lacking the CC genotype.

The detection step in the present method is carried out by, for example, allele-specific probe hybridization, allele-specific primer extension, allele-specific amplification, sequencing, 5′ nuclease digestion, molecular beacon assay, oligonucleotide ligation assay, restriction fragment size analysis, invasive cleavage assay, branch migration assay, denaturing gradient gel electrophoresis, immunoassay, or single-stranded conformation polymorphism analysis.

In certain embodiments, the human is treated with the topical beta-blocker timolol, levobunolol, betaxolol, metipranolol, carteolol, timolol combined with dorzolamide, or timolol combined with brimonidine

In another aspect, the invention provides a method of genotype-based glaucoma management to reduce intraocular pressure in a glaucoma-afflicted human. Such a method includes steps of: (a) detecting a CC genotype at coding single nucleotide polymorphism (SNP) rs1042714 in the ADRB2 gene (Gln27Glu) in a nucleic acid sample from the human, wherein the presence of the CC genotype indicates that the glaucoma-afflicted human will experience a clinically meaningful reduction in intraocular pressure upon treatment with a topical beta-blocker as compared to a glaucoma-afflicted human lacking the CC genotype and undergoing the same treatment; and (b) administering topical beta-blocker to the glaucoma-afflicted human in which was detected the CC genotype at coding single nucleotide polymorphism (SNP) rs1042714 in the ADRB2 gene (Gln27Glu) to thereby provide a clinically meaningful reduction in intraocular pressure in the glaucoma-afflicted human.

In yet another aspect, the invention encompasses a kit for identifying a glaucoma-afflicted human who will experience a clinically meaningful reduction in intraocular pressure upon treatment with a topical beta-blocker. Such a kit includes: (a) a detection probe for detecting a CC genotype at coding single nucleotide polymorphism (SNP) rs1042714 in the ADRB2 gene (Gln27Glu) in a nucleic acid sample from a human, wherein the presence of the CC genotype indicates that the glaucoma-afflicted human will experience a clinically meaningful reduction in intraocular pressure upon treatment with a topical beta-blocker as compared to a glaucoma-afflicted human lacking the CC genotype and undergoing the same treatment; (b) one or more nucleic acids that serve as controls for the detection probe; and (c) instructional material for interpreting results obtained by use of the kit for the prediction of intraocular pressure reduction following treatment with a beta-blocker.

In certain embodiments, the kit further includes at least one pair of amplification primers, the primers designed to bind respective nucleic acid sequences upstream and downstream of the SNP thereby flanking the SNP rs1042714 in the ADRB2 gene.

In exemplary embodiments, the detection probe is an allele specific detection probe labeled with a chromogenic, radioactive, or a luminescent moiety. The detection probe is preferably labeled with a fluorescent moiety.

These and other features, objects and advantages of the present invention will become better understood from the description that follows. The description of preferred embodiments is not intended to limit the invention to cover all modifications, equivalents and alternatives. Reference should therefore be made to the claims recited herein for interpreting the scope of the invention.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

In describing the present invention, the following terms will be employed and are defined as indicated below.

A “single nucleotide polymorphism” or “SNP” refers to a variation in the nucleotide sequence of a polynucleotide that differs from another polynucleotide by a single nucleotide difference. For example, without limitation, exchanging one A for one C, G or T in the entire sequence of polynucleotide constitutes a SNP. It is possible to have more than one SNP in a particular polynucleotide. For example, at one position in a polynucleotide, a C may be exchanged for a T, at another position a G may be exchanged for an A and so on. When referring to SNPs, the polynucleotide is most often DNA. The term “allele” refers to one or more alternative forms of a particular sequence that contains a SNP. The sequence may or may not be within a gene.

The terms “subject”, “patient” and “individual” refer to a human being.

A “glaucoma-afflicted human” refers to a human suffering from glaucoma. Glaucoma is an ocular disease characterized by changes in the optic nerve and the field of vision, and absence of other known optic nerve disease. The optic neuropathy characteristic of glaucoma is seen clinically as an enlargement of the size of the optic cup, thinning of the neuroretinal rim, disc hemorrhage, and nerve fiber layer defects. Glaucoma is often associated with an elevated intraocular pressure.

A “clinically meaningful reduction in intraocular pressure” refers to at least a 20% reduction in intraocular pressure (“IOP”) observed between temporally-spaced IOP measurements conducted on a glaucoma-afflicted human.

The term “topical beta-blocker” shall mean a class of medications which blocks beta adrenergic agonist receptors in the eye and is useful to lower intraocular pressure, particularly through administration in eye drop form to the affected eye. Examples of such topical medications as single agent therapy include timolol, levobunolol, betaxolol, metipranolol, carteolol. Examples as combination therapy include Cosopt (timolol combined with dorzolamide) and Combigan (timolol combined with brimonidine).

“Treatment with a topical beta-blocker” shall minimally refer to at least a single administration of topical beta-blocker to an affected eye of a glaucoma-afflicted human. In certain treatment strategies, the course of administration is to administer one drop in the affected eye once or twice daily until the patient returns for evaluation of response, at which time the medication may be continued on a long term basis or discontinued due to lack of clinically meaningful reduction in intraocular pressure.

“Amplification” refers to any means by which a polynucleotide sequence is copied and thus expanded into a larger number of polynucleotide sequences, e.g., by reverse transcription, polymerase chain reaction or ligase chain reaction, among others.

As used herein, an “instructional material” includes a publication, a recording, a diagram, or any other medium of expression which can be used to communicate the usefulness of the kit for its designated use in practicing a method of the invention. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

An “isolated” polynucleotide or polypeptide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “nucleic acid” typically refers to large polynucleotides. A “polynucleotide” means a single strand or parallel and anti-parallel strands of a nucleic acid. Thus, a polynucleotide may be either a single-stranded or a double-stranded nucleic acid. A polynucleotide is not defined by length and thus includes very large nucleic acids, as well as short ones, such as an oligonucleotide The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”. Sequences on a DNA strand which are located 5′ to a reference point on the DNA are referred to as “upstream sequences”. Sequences on a DNA strand which are 3′ to a reference point on the DNA are referred to as “downstream sequences.”

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. Typical uses of primers include, but are not limited to, sequencing reactions and amplification reactions. A primer is typically single-stranded, but may be double-stranded. Primers are typically deoxyribonucleic acids, but a wide variety of synthetic and naturally-occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., detectable moieties, such as chromogenic, radioactive or fluorescent moieties, or moieties for isolation, e.g., biotin.

“Probe” refers to a polynucleotide that is capable of specifically hybridizing to a designated sequence of another polynucleotide. “Probe” as used herein encompasses oligonucleotide probes. A probe may or may not provide a point of initiation for synthesis of a complementary polynucleotide. A probe specifically hybridizes to a target complementary polynucleotide, but need not reflect the exact complementary sequence of the template. In such a case, specific hybridization of the probe to the target depends on the stringency of the hybridization conditions. For use in SNP detection, some probes are allele-specific, and hybridization conditions are selected such that the probe binds only to a specific SNP allele. Probes can be labeled with, e.g., detectable moieties, such as chromogenic, radioactive or fluorescent moieties, and used as detectable agents.

As used herein, “label” refers to a group covalently attached to a polynucleotide. The label may be attached anywhere on the polynucleotide but is preferably attached at one or both termini of the polynucleotide. The label is capable of conducting a function such as giving a signal for detection of the molecule by such means as fluorescence, chemiluminescence, and electrochemical luminescence. Alternatively, the label allows for separation or immobilization of the molecule by a specific or non-specific capture method (Andrus, 1995, In: PCR 2: A Practical Approach, McPherson et al. (eds) Oxford University Press, Oxford, England, pp. 39-54). Labels include, but are not limited to, fluorescent dyes, such as fluorescein and rhodamine derivatives (U.S. Pat. Nos. 5,188,934; 5,366,860), cyanine dyes, haptens, and energy-transfer dyes (Clegg, 1992, Meth. Enzymol. 211:353-388; Cardullor et al., 1988, PNAS 85:8790-8794).

The term “capable of hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of a nucleic acid or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g., low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65 degrees Celsius in an aqueous solution, followed by washing with 1×SSC at 65 degrees C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

Typically, stringent conditions will be those in which the salt concentration is less than about 1.5 M sodium ion, typically about 0.01 to 1.0 M Na ion concentration (or other salts) from about pH 7.0 to about pH 8.3 and the temperature is at least about 30 degrees Celsius for short probes (e.g., 10 to 50 nucleotides) and at least about 60 degrees C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37 degrees Celsius, and a wash in 1-2×SSC at 50 to 55 degrees Celsius. Exemplary moderate stringency conditions include hybridization in 40 to 45% formamide, 1 M NaCl, 1% SDS at 37 degrees Celsius, and a wash in 0.5-1×SSC at 55 to 60 degrees Celsius. Exemplary high stringency conditions include hybridization in 50% formamide, 1 M NaCl, 1% SDS at 37 degrees Celsius, and a wash in 0.1×SSC at 60 to 65 degrees Celsius.

Methods and materials of the invention may be used more generally to evaluate a DNA sample from an individual, genetically type an individual, and detect genetic differences between individuals. In particular, a sample of genomic DNA from an individual may be evaluated by reference to one or more controls to determine if a SNP, or group of SNPs, in a gene is present. Any method for determining genotype can be used for determining the genotype in the present invention. Such methods include, but are not limited to, DNA sequencing, fluorescence spectroscopy, fluorescence resonance energy transfer (or “FRET”)-based hybridization analysis, high throughput screening, mass spectroscopy, microsatellite analysis, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties.

A “restriction fragment” refers to a fragment of a polynucleotide generated by a restriction endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

In the effort providing the present invention, the inventors examined medical records of 18,773 adults to extract all intraocular pressure (IOP) measurements for subjects who had been prescribed a topical beta-blocker. Five single nucleotide polymorphisms (SNPs) in the beta-1, beta-2, and beta-3 adrenergic receptor genes were genotyped, and six polymorphisms in the CYP2D6 gene were genotyped. A total of 58.1% of the subjects were female; mean age 63.8 years. Topical beta-blockers were prescribed for 343 eyes of 215 subjects. A ≧20% IOP reduction in one or both eyes was observed in 61.0% of subjects. Males were significantly more likely than females to have a ≧20% IOP drop (69.3% versus 54.9%, chi-squared=4.5, P=0.04). After adjusting for gender, family history of glaucoma and use of systemic beta-blockers, subjects with the CC genotype at coding SNP rs1042714 in the ADRB2 gene were significantly more likely to experience a ≧20% decrease in IOP (OR=2.0, 95% CI=1.00-4.02). As can be appreciated, genotype-based drug prescribing according to the present invention provides means for improved glaucoma management along with significant reductions in healthcare costs.

The present inventors identified that candidate pharmacodynamic polymorphisms were associated with IOP response to topical beta-blockers. Specifically, a coding SNP in the ADRB2 gene (Gln27Glu) was associated with two-fold greater odds of a clinically meaningful reduction in IOP following treatment with a topical beta-blocker.

The beta-adrenergic receptor is a member of the adrenergic family of G-protein coupled receptors. Epinephrine and norepinephrine are the primary endogenous agonists, but other endogenous catecholamines (e.g., dopamine) can interact with these receptors as well. In the early 1990s investigators characterized several ADRB2 polymorphisms with altered signaling properties. In 1999, an ADRB1 (SEQ. ID. NOs: 3-4) polymorphism was reported which altered the cytoplasmic tail near the seventh transmembrane spanning segment. Using site directed mutagenesis, investigators were able to show that the resulting amino acid change (Gly389Arg) in ADRB1 was associated with differential adenylate cyclase activation in permanently transfected fibroblasts (CHW-1102 cells). Other investigators have subsequently linked this ADRB1 SNP to altered receptor expression. Two non-synonymous coding SNPs in ADRB2 have also been associated with altered cellular receptor trafficking (in vitro). Importantly, these SNPs have also been associated (in vivo) with altered clinical outcomes, including asthma and acute coronary syndromes. Further, there appears to be marked linkage disequilibrium between these two ADRB2 SNPs.

Previous studies have looked at the beta-2 adrenergic receptor gene in relation to glaucoma and IOP, with varying results, some differing from the results described herein. The differing results between the present study and the previous studies of beta-2 adrenergic receptor gene and IOP could be due to the differing study populations or a lack of statistical power in the previous studies.

In one particular study of 505 Japanese subjects, the IOP at glaucoma diagnosis was found to be significantly higher in patients carrying 27Glu. This coding SNP (C79G transversion) induces a non-conservative amino acid substitution (Gln27Glu) near the N-terminus of the ADRB2 gene product. The result is an alteration in agonist activity which promoted down-regulation of the receptor. Additional in vitro studies have demonstrated that Gln27Glu affects receptor function. A 60-fold greater isoprenaline concentration has been previously shown necessary to down-regulate Glu27 to the same extent as Gln27.

The inventors' success in arriving at the present invention was due in large part to the population-based nature of the study cohort, which allowed inferences to the entire population to be made.

As can be appreciated, the inventors have found that a coding SNP in the ADRB2 gene is associated with increased likelihood of a clinically meaningful IOP response to topical beta-blockers. Topical beta-blockers are the least expensive agent used to treat glaucoma and ocular hypertension, and therefore genotyped-based prescribing will provide improved glaucoma regimens to patients along with significant savings in health care costs.

Accordingly, in a first aspect of the invention, a certain single nucleotide polymorphism (SNP) is provided which is associated with an glaucoma-afflicted individual's genetic predisposition to treatment by topical beta-blockers. Therefore, the present invention provides nucleic acids and methods useful to determine the presence of the SNP in glaucoma-afflicted individuals for the purpose of genotype-based drug prescribing. Kits useful in practicing embodiments of the inventive methods are also provided.

It is commonly understood that specific sites in the human genomic DNA sequence are polymorphic, i.e., within a population, more than one nucleotide (G, A, T, C) is found at a specific position. These SNPs are often useful to detect genetic linkage to phenotypic variation in activity and expression of a particular protein, as in the present case.

SNPs are generally biallelic systems, that is, there are two alleles that an individual may have for any particular marker. SNPs, found approximately every kilobase, offer the potential for generating very high density genetic maps, which are extremely useful for developing haplotyping systems for genes or regions of interest, and because of the nature of SNPs, they may in fact be the polymorphisms associated with the disease phenotypes under study. The low mutation rate of SNPs also makes them excellent markers for studying complex genetic traits.

In order to provide an unambiguous identification of the specific site of a polymorphism, sequences flanking the polymorphic site may be shown and/or described herein, where the 5′ and 3′ flanking sequence is non-polymorphic, and the central position is variable. It will be understood that there is no special significance to the length of non-polymorphic flanking sequence that is included, except to aid in positioning the polymorphism in the genomic sequence.

Nucleic acids particularly relative to the present invention comprise the provided variant nucleotide sequence encoding the CC genotype at coding SNP rs1042714 in the human ADRB2 gene (SEQ. ID NOs: 1-2, Genbank accession number NM_{—}000024.4 (mRNA); Genbank accession number NC_{—}000005.8 (chromosome 5); Genbank accession number NP_{—}000015.1 (protein); chromosomal position 148186349 . . . 148188381; described at Rehsaus et al, Mutations in the gene encoding for the (β₂-adrenergic receptor in normal and asthmatic subjects. Am J Respir Cell Mol Biol 8:334-339 (1993); all accession and literature citations incorporated herein by reference in their entirety). As described herein, nucleic acids acting as hybridization probes may be used where differing polymorphic forms are present, either in separate reactions, or labeled such that they can be distinguished from each other. Assays may utilize nucleic acids that hybridize to one or more of the described polymorphisms.

Nucleic acids including the SNP of interest may be obtained by chemically synthesizing oligonucleotides in accordance with conventional methods, by restriction enzyme digestion, by PCR amplification, etc. For the most part, useful DNA fragments will be of at least 15 nt, usually at least 20 nt, often at least 50 nt. Such small DNA fragments are useful as primers for PCR, hybridization screening, etc. Larger DNA fragments, i.e. greater than 100 nt are useful for production of the encoded polypeptide, promoter motifs, etc. For use in amplification reactions, such as PCR, a pair of primers will be used. The exact composition of primer sequences is not critical to the invention, but for most applications, the primers will hybridize to the subject sequence under stringent conditions, as defined herein.

Nucleic acid sequences containing the relevant SNP are isolated and obtained in substantial purity, generally as other than an intact mammalian chromosome. Usually, the DNA will be obtained substantially free of other nucleic acid sequences that do not include the present sequences or fragments thereof, generally being at least about 50%, usually at least about 90% pure and are typically “recombinant”, i.e. flanked by one or more nucleotides with which it is not normally associated on a naturally occurring chromosome.

Vectors useful for introduction of a nucleic acid containing the SNP of interest include plasmids and viral vectors, e.g. retroviral-based vectors, adenovirus vectors, etc. that are maintained transiently or stably in mammalian cells. A wide variety of vectors can be employed for transfection and/or integration of nucleic acid into the genome of the cells. Alternatively, micro-injection may be employed, fusion, or the like for introduction of nucleic acids into a suitable host cell.

In another aspect, the present invention is directed to the identification of the CC genotype at coding SNP rs1042714 in the human ADRB2 gene for the purpose of identifying a glaucoma-afflicted individual having greater odds, preferably more then two-fold, of a clinically meaningful reduction in IOP following treatment with a topical beta-blocker. Such a method is carried out on a sample obtained from the glaucoma-afflicted individual.

Biological samples useful in the practice of the methods of the invention can be any biological sample from which any of genomic DNA, mRNA, unprocessed RNA transcripts of genomic DNA or combinations of the three can be isolated. As used herein, “unprocessed RNA” refers to RNA transcripts which have not been spliced and therefore contain at least one intron. Suitable biological samples include, but are not limited to, blood, buccal swabs, hair, bone, and tissue samples, such as skin or biopsy samples. Biological samples also include cell cultures established from an individual.

Genomic DNA, mRNA, and/or unprocessed RNA transcripts are isolated from the biological sample by conventional means known to the skilled artisan. See, for instance, Sambrook et al. (2001, Molecular Cloning: A Laboratory Manual, Cold Spring Harbor Laboratory Press, Cold Spring Harbor, N.Y.) and Ausubel et al. (eds., 1997, Current Protocols in Molecular Biology, John Wiley & Sons, New York). The isolated genomic DNA, mRNA, and/or unprocessed RNA transcripts is used, with or without amplification, to detect a SNP of the invention.

Many SNP identification methods that can be used in the methods of the invention involve amplifying a target polynucleotide sequence prior to detecting the SNP identity. A “target polynucleotide sequence” is a region of the genomic DNA, mRNA or unprocessed RNA containing the SNP of interest. Some methods, including the 5′ nuclease assay described herein, combine the amplification and detection processes in one step, as described elsewhere herein. Other methods, such as the invasive cleavage assay also described herein, use signal amplification and are thereby sufficiently sensitive such that the genomic nucleic acid sample does not need to be amplified.

Amplification of a target polynucleotide sequence may be carried out by any method known to the skilled artisan. See, for instance, Kwoh et al., (1990, Am. Biotechnol. Lab. 8, 14-25) and Hagen-Mann, et al., (1995, Exp. Clin. Endocrinol. Diabetes 103:150-155). Amplification methods include, but are not limited to, polymerase chain reaction (“PCR”) including RT-PCR, strand displacement amplification (Walker et al., 1992, PNAS 89: 392-396; Walker et al., 1992, Nucleic Acids Res. 20: 1691-1696), strand displacement amplification using Phi29 DNA polymerase (U.S. Pat. No. 5,001,050), transcription-based amplification (Kwoh et al., 1989, PNAS 86: 1173-1177), self-sustained sequence replication (“3SR”) (Guatelli et al., 1990, PNAS 87: 1874-1878; Mueller et al., 1997, Histochem. Cell Biol. 108:431-437), the Q.beta. replicase system (Lizardi et al., 1988, BioTechnology 6: 1197-1202; Cahill et al., 1991, Clin., Chem. 37:1482-1485), nucleic acid sequence-based amplification (“NASBA”) (Lewis, 1992, Genetic Engineering News 12 (9), 1), the repair chain reaction (“RCR”) (Lewis, 1992, supra), and boomerang DNA amplification (or “BDA”) (Lewis, 1992, supra). PCR is the preferred method of amplifying the target polynucleotide sequence.

PCR may be carried out in accordance with known techniques. See, e.g., Bartlett et al., eds., 2003, PCR Protocols Second Edition, Humana Press, Totowa, N.J. and U.S. Pat. Nos. 4,683,195; 4,683,202; 4,800,159; and 4,965,188. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained. The amplified target polynucleotide may be used in one of the detection assays described elsewhere herein to identify the SNP present in the amplified target polynucleotide sequence.

Nucleic acid amplification techniques, such as the foregoing, and SNP allele detection methods, as described below, may involve the use of a primer, a pair of primers, or two pairs of primers which specifically bind to nucleic acid containing the SNP to be detected, and do not bind to nucleic acid that does not contain the SNP to be detected under the same hybridization conditions. Such probes are sometimes referred to as “amplification primers” herein.

In some detection assays, a polynucleotide probe, which is used to detect DNA containing a SNP of interest, is a probe which binds to DNA encoding a specific SNP allele, but does not bind to DNA that does not encode that specific SNP allele under the same hybridization conditions. For instance, the detection probe used for 5′ nuclease assay, described herein, straddles a SNP site and discriminates between alleles. In other assays, a polynucleotide probe which is used to detect DNA containing a SNP of interest is a probe that binds to either SNP allele at a sequence that does not include the SNP. This type of probe may bind to a sequence immediately 3′ to the SNP or may bind to a sequence that is 3′ to the SNP and removed from the SNP by one or more bases. In some cases, the polynucleotide probe is labelled with one or more labels, such as those, for instance, set forth elsewhere herein in the 5′ nuclease assay. Polynucleotide probes as described above are sometimes referred to as “detection probes” or “detection primers” herein.

Probes and primers may be any suitable length, but are typically oligonucleotides from 5, 6, 8 or 12 nucleotides in length up to 40, 50 or 60 nucleotides in length, or more. The oligonucleotide typically comprises a region of nucleotide sequence that hybridizes under stringent conditions to at least about 5, 6, 8, 12, 20, 25, 40, 50 or more consecutive nucleotides in the target polynucleotide sequence. The skilled artisan knows where the region of consecutive nucleotides intended to hybridize to the target polynucleotide sequence must be located in the oligonucleotide, based on the intended use of the oligonucleotide. For instance, in an oligonucleotide for use in a primer extension assay, the skilled artisan knows the region of consecutive nucleotides must include the 3′ terminal nucleotide. The probes and primers are typically substantially purified. Such probes and/or primers may be immobilized on or coupled to a solid support such as a bead, glass slide or chip in accordance with known techniques, and/or coupled to or labelled with a detectable label such as a fluorescent compound, a chemiluminescent compound, a radioactive element, or an enzyme in accordance with known techniques.

Probes and primers are designed using the sequences flanking the SNP in the target polynucleotide sequence. Depending on the particular SNP identification protocol utilized, the consecutive nucleotides of the region that hybridizes to a target polynucleotide sequence may include the target SNP position. Alternatively the region of consecutive nucleotides may be complementary to a sequence in close enough proximity 5′ and/or 3′ to the SNP position to carry out the desired assay. The skilled artisan can readily design primer and probe sequences using the sequences provided herein. Considerations for primer and probe design with regard to, for instance, melting temperature and avoidance of primer-dimers, are well known to the skilled artisan. In addition, a number of computer programs, such as Primer Express. (Applied Biosystems, Foster City, Calif.) and Primo SNP 3.4 (Chang Bioscience, Castro Valley, Calif.), can be readily used to obtain optimal primer/probe sets. The probes and primers may be chemically synthesized using commercially available reagents and synthesizers by methods that are well-known in the art (see, e.g., Herdwijn, 2004, Oligonucleotide Synthesis: Methods and Applications, Humana Press, Totowa, N.J.).

The process of identifying the nucleotide present at the SNP positions disclosed herein is referred to by phrases including, but not limited to: “SNP identification”, “SNP genotyping”, “SNP typing”, “SNP detection” and “SNP scoring”.

The method of the invention can identify a nucleotide occurrence for either the plus or minus strand of DNA. That is, the invention encompasses not only identifying the nucleotide at the SNP position in the strand, but also identifying the nucleotide at the SNP position in the corresponding complementary minus strand. For instance, for a SNP in which the allele associated with an elevated risk of a disease or malady has a “C” at the SNP on the plus strand, detecting a “G” in the SNP position of the complementary, minus strand is also indicative of that same elevated risk of disease or malady.

There are numerous methods of SNP identification known to the skilled artisan. See, for instance, Kwok (2001, Annu Rev. Genomics Hum. Genet. 2:235-258) and Theophilus et al., (2002, PCR Mutation Detection Protocols, Humana Press, Totowa, N.J.). Any may be used in the practice of the present invention. SNP identification methods include, but are not limited to, 5′ nuclease assay, primer extension or elongation assays, allele specific oligonucleotide ligation, allele specific hybridization, sequencing, invasive cleavage reaction, branch migration assay, single strand conformational polymorphism (SSCP), denaturing gradient gel electrophoresis (DGGE) and immunoassay. Many of these assays have or can be adapted for microarrays. See, for instance, Erdogan et al. (2001, Nuc. Acids Res. 29:e36); O'Meara et al. (2002, Nuc. Acids Res. 30:e75); Pastinen et al. (1997, Genome Res. 7:606-614); Pastinen et al. (2000, Genome Res. 10:1031-1042); and U.S. Pat. No. 6,294,336. Preferred SNP genotyping methods are the 5′ nuclease assay, primer extension assays and sequencing.

The 5′ nuclease assay, also known as the 5′ nuclease PCR assay and the TaqMan Assay (Applied Biosystems, Foster City, Calif.), provides a sensitive and rapid means of genotyping SNPs. The 5′ nuclease assay detects, by means of a probe, the accumulation of a specific amplified product during PCR. The probe is designed to straddle a target SNP position and hybridize to the target polynucleotide sequence containing the SNP position only if a particular SNP allele is present. During the PCR reaction, the DNA polymerase, which extends an amplification primer annealed to the same strand and upstream of the hybridized probe, uses its 5′ nuclease activity and cleaves the hybridized probe. There are different ways to detect the probe cleavage. In one common variation, the 5′ nuclease assay utilizes an oligonucleotide probe labeled with a fluorescent reporter dye at the 5′ end of the probe and a quencher dye at the 3′ end of the probe. See, for instance, Lee et al., (1993), Nuc. Acids Res. 21:3761-3766), Livak (1999, Genet. Anal. 14:143-149) and U.S. Pat. Nos. 5,538,848, 5,876,930, 6,030,787, 6,258,569 and 6,821,727. The proximity of the quencher dye to the fluorescent reporter in the intact probe maintains a reduced fluorescence for the reporter. Cleavage of the probe separates the fluorescent reporter dye and the quencher dye, resulting in increased fluorescence of the reporter. The 5′ nuclease activity of DNA polymerase cleaves the probe between the reporter and the quencher only if the probe hybridizes to the target, and the target is amplified during PCR. Accumulation of a particular PCR product is thus detected directly by monitoring the increase in fluorescence of the reporter dye. In another variation, the oligonucleotide probe for each SNP allele has a unique fluorescent dye and detection is by means of fluorescence polarization (Kwok, 2002, Human Mutat. 19:315-323). This assay advantageously can detect heterozygotes.

The primer extension reaction (also called “mini-sequencing”, “single base extension assay” or “single nucleotide extension assay”, and “primer elongation assay”) involves designing and annealing a primer to a sequence downstream of a target SNP position in an amplified target polynucleotide sequence (“amplified target”). A mix of dideoxynucleotide triphosphates (ddNTPs) and/or deoxynucleotide triphosphates (dNTPs) are added to a reaction mixture containing amplified target, primer, and DNA polymerase. Extension of the primer terminates at the first position in the PCR amplified target where a nucleotide complementary to one of the ddNTPs in the mix occurs. The primer can be annealed to a sequence either immediately 3′ to or several nucleotides removed from the SNP position. For single base or single nucleotide extension assays, the primer is annealed to a sequence immediately 3′ the SNP position. If the primer anneals to a sequence several nucleotides removed from the target SNP, the only limitation is that the template sequence between the 3′ end of the primer and the SNP position can not contain a nucleotide of the same type as the one to be detected, or this will cause premature termination of the extension primer. Alternatively, if all four ddNTPs alone, and no dNTPs, are added to the reaction mixture, the primer will always be extended by only one nucleotide, corresponding to the target SNP position. In this instance, primers are designed to bind to a sequence one nucleotide downstream from the SNP position. In other words, the nucleotide at the 3′ end of the primer hybridizes to the nucleotide immediately 3′ to the SNP position. Thus, the first nucleotide added to the primer is at the SNP. In one common variation, the ddNTPs used in the assay each have a unique fluorescent label, enabling the detection of the specific nucleotide added to the primer. SNaPshot from Applied Biosystems is a commercially available kit for single nucleotide primer extension using fluorescent ddNTPs, and can be multiplexed. SNP-IT™ (Orchid Cellmark, Princeton, N.J.) is another commercially available product using a primer extension assay for identifying SNPs (see also U.S. Pat. No. 5,888,819). Some variations of the primer extension assay can identify heterozygotes.

An alternate detection method uses mass spectrometry to detect the specific nucleotide added to the primer in a primer extension assay. See, for instance, Haffet al. (1997, Genome Res. 7:378-388). Mass spectrometry (“mass spec”) takes advantage of the unique mass of each of the four nucleotides of DNA. SNPs can be unambiguously genotyped based on the slight differences in mass, and the corresponding time of flight differences, inherent in nucleic acid molecules having different nucleotides at a single base position. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry.

For detection by mass spectrometry, extension by only one nucleotide is preferable, as it minimizes the overall mass of the extended primer, thereby increasing the resolution of mass differences between alternative SNP nucleotides. Furthermore, mass-tagged dideoxynucleoside triphosphates (ddNTPs) can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer. The primers are extended, purified and then analyzed by MALDI-TOF mass spectrometry to determine the identity of the nucleotide present at the SNP position. MassARRAY™ (Sequenom, San Diego, Calif.) is a commercially available system for SNP identification using mass spectrometry.

The primer extension assay has also been modified to use fluorescence polarization as the means of detecting the specific nucleotide at the SNP position. This modified assay is sometimes referred to as template-directed dye-terminator incorporation assay with fluorescence polarization (FP-TDI). See Kwok (2002, supra). A kit for this assay, Acycloprimer™-FP, is commercially available from Perkin Elmer (Boston, Mass.).

Allele-specific oligonucleotide ligation, also called oligonucleotide ligation assay (OLA) and is similar in many respects to ligase chain reaction, uses a pair of oligonucleotide probes that hybridize to adjacent segments of sequence on a nucleic acid fragment containing the SNP. One of the probes has a SNP allele-specific base at its 3′ or 5′ end. The second probe hybridizes to sequence that is common to all SNP alleles. If the first probe has an allele-specific base at its 3′ end, the second probe hybridizes to the sequence segment immediately 3′ to the SNP. If the first probe has an allele-specific base at its 5′ end, the second probe hybridizes to the sequence segment immediately 5′ to the SNP. The two probes can be ligated together only when both are hybridized to a DNA fragment containing the SNP allele for which the first probe is specific. See Landegren et al. (1988, Science 241:1077-80). One method of detecting the ligation product involves fluorescence. The second probe, which hybridizes to either allele, is fluorescently labeled. The allele-specific probe is labeled with biotin. Strepavidin capture of the allele-specific ligation product and subsequent fluorescent detection is used to determine which SNP is present. Another variation of this assay combines amplification and ligation in the same step (Barany, 1991, PNAS 88:189-93). A commercially available kit, SNPlex™ (Applied Biosystems, Foster City, Calif.) uses capillary electrophoresis to analyze the ligation products.

Allele-specific hybridization, also called allele-specific oligonucleotide hybridization (ASO), distinguishes between two DNA molecules differing by one base using hybridization. Amplified DNA fragments containing the target SNP are hybridized to allele-specific oligonucleotides. In one variation, the amplified DNA fragments are fluorescence labeled and the allele-specific oligonucleotides are immobilized. See, for instance, Strachan et al., (1999, In: Human Molecular Genetics, Second Edition, John Wiley & Sons, New York, N.Y.). In another variation, the allele-specific oligonucleotides are labeled with a antigen moiety. Binding is detected via an enzyme-linked immunoassay and color reaction (see, for instance, Knight et al., 1999, Clin. Chem. 45:1860-1863). In yet another variation, the allele-specific oligonucleotides are radioactively labeled (see, for instance, Saiki et al., 1986, Nature 324:163-6). Protein nucleic acid (PNA) probes and mass spec may also be used (Ross et al., 1997, Anal. Chem. 69:4197-4202).

Allele-specific hybridization may also be performed by using an array of oligonucleotides, where discrete positions on the array are complementary to one or more of the provided polymorphic sequences, e.g. oligonucleotides of at least 12 nt, frequently 20 nt, or larger, and including the sequence flanking the polymorphic position. Such an array may comprise a series of oligonucleotides, each of which can specifically hybridize to a different polymorphism. For examples of arrays, see Hacia et al. (1996) Nature Genetics 14:441-447; Lockhart et al. (1996) Nature Biotechnol. 14:1675-1680; and De Risi et al. (1996) Nature Genetics 14:457-460.

Other SNP identification methods based on the formation of allele-specific complexes include the invasive cleavage assay and the branch migration assay. The invasive cleavage assay uses two probes that have a one nucleotide overlap. When annealed to target DNA containing the SNP, the one nucleotide overlap forms a structure that is recognized by a 5′ nuclease that cleaves the downstream probe at the overlap nucleotide. The cleavage signal can be detected by various techniques, including fluorescence resonance energy transfer (FRET) or fluorescence polarization. Reaction conditions can be adjusted to amplify the cleavage signal, allowing the use of very small quantities of target DNA. Thus, the assay does not require amplication of the target prior to detecting the SNP identity, although an amplified sequence may be used. See, for instance, Lyamichev et al., 2003, Methods Mol. Biol 212:229-240; Brookes, 1999, Gene, 234:177-186; and Mein et al., 2000, Genome Res. 10:330-343). A commercially available product, the Invader® assay (Third Wave Molecular Diagnostics, Madison, Wis.), is based on this concept. The branch migration assay based on Holliday junction migration, involves the detection of a stable four-way complex for SNP identification (See, for instance, U.S. Pat. No. 6,878,530).

SNPs can also be scored by direct DNA sequencing. A variety of automated sequencing procedures may be utilized when performing the diagnostic assays (Naeve et al., 1995, Biotechniques 19:448-453), including sequencing by mass spectrometry (see, e.g., PCT International Publication No. WO 94/16101; Cohen et al., 1996, Adv. Chromatogr. 36:127-162; and Griffin et al., 1993, Appl. Biochem. Biotechnol. 38:147-159). Traditional sequencing methods may also be used, such as dideoxy-mediated chain termination method (Sanger et al., 1975, J. Molec. Biol. 94: 441; Prober et al. 1987, Science 238: 336-340) and the chemical degradation method (Maxam et al., 1977, PNAS 74: 560).

A preferred sequencing method for SNPs is pyrosequencing. See, for instance, Ahmadian et al., 2000, Anal. Biochem, 280:103-110; Alderborn et al., 2000, Genome Res. 10:1249-1258 and Fakhrai-Rad et al., 2002, Hum. Mutat. 19:479-485. Pyrosequencing involves a cascade of four enzymatic reactions that permit the indirect luciferase-based detection of the pyrophosphate released when DNA polymerase incorporates a dNTP into a template-directed growing oligonucleotide. Each dNTP is added individually and sequentially to the same reaction mixture, and subjected to the four enzymatic reactions. Light is emitted only when a dNTP is incorporated, thus signaling which dNTP in incorporated. Unincorporated dNTPs are degraded by apyrase prior to the addition of the next dNTP. The method can detect heterozygous individuals in addition to heterozygotes. Pyrosequencing uses single stranded template, typically generated by PCR amplification of the target sequence. One of the two amplification primers is biotinylated thereby enabling streptavidin capture of the amplified duplex target. Streptavidin-coated beads are useful for this step. The captured duplex is denatured by alkaline treatment, thereby releasing the non-biotinylated strand. The detection primer used for SNP identification using pyrosequencing is designed to hybridize to a sequence 3′ to the SNP. In a preferred embodiment, the 3′ sequence is immediately adjacent to the SNP position. Thus, the SNP identity is ascertained when the first nucleotide is incorporated. Pyrosequencing can detect heterozygotes.

Further examples of methods that can be used to identify for the SNPs of the present invention include single-strand conformational polymorphism (SSCP) and denaturing gradient gel electrophoresis (DGGE). SSCP identifies base differences by alteration in electrophoretic migration of single stranded PCR products, as described in Orita et al., (1989, PNAS 86:2766-1770). Single-stranded PCR products can be generated by heating or otherwise denaturing double-stranded PCR products. Single-stranded nucleic acids may refold or form secondary structures that are partially dependent on the base sequence. The different electrophoretic mobilities of single-stranded amplification products. are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Myers et al., 1985, Nature 313:495 and Erlich, ed., 1992, In: PCR Technology, Principles and Applications for DNA Amplification, W. H. Freeman and Co, New York, Chapter 7).

Sequence-specific ribozymes (U.S. Pat. No. 5,498,531) can be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis. Immunoassay methods using antibodies specific for SNP alleles can be used for SNP detection. Southern and Northern blot analysis can also be utilized for nucleic acid analysis. See, for instance, Sambrook et al. (2001, Molecular Cloning: A Laboratory Manual, Cold Spring Harbor Laboratory Press, Cold Spring Harbor, N.Y.), Ausubel et al. (eds., 1997, Current Protocols in Molecular Biology, John Wiley & Sons, New York), and Gerhardt et al. (eds., 1994, Methods for General and Molecular Bacteriology, American Society for Microbiology, Washington, D.C.).

The invention encompasses diagnostic screening using the present SNP genetically linked to a phenotypic variant in activity or expression. Two polymorphic variants may be in linkage disequilibrium, i.e. where alleles show non-random associations between genes even though individual loci are in Hardy-Weinberg equilibrium. Linkage analysis may be performed alone, or in combination with direct detection of phenotypically evident polymorphisms. The use of SNPs for genotyping is illustrated in Golevleva et al., (1996, Am. J. Hum. Genet. 59:570-578;) and in Underhill et al. (1996, PNAS, 93:196-200).

The invention also provides a kit useful in practicing the method of the invention. The kit may contain at least one pair of amplication primers that is used to amplify a target polynucleotide sequence containing the SNP relevant to the present invention. The amplification primers are designed based on the sequences provided herein for the upstream and downstream sequence flanking the SNP. In a preferred embodiment, the amplification primers will generate an amplified double-stranded target polynucleotide between about 50 base pairs to about 600 base pairs in length and, more preferably, between about 100 base pairs to about 300 base pairs in length. In another preferred embodiment, the SNP is located approximately in the middle of the amplified double-stranded target polynucleotide.

The kit may further contain a detection probe designed to hybridize to a sequence 3′ to the SNP on either strand of the amplified double-stranded target polynucleotide. In one variation, the detection probe hybridizes to the sequence immediately 3′ to the SNP on either strand of the amplified double-stranded target polynucleotide but does not include the SNP. This kit variation may be used to identify the SNP by pyrosequencing or a primer extension assay. For use in pyrosequencing, one of the amplification primers in the kit may be biotinylated and the detection probe is designed to hybridize to the biotinylated strand of the amplified double-stranded target polynucleotide. For use in a primer extension assay, the kit may optionally also contain fluorescently labeled ddNTPs. Typically, each ddNTP has a unique fluorescent label so they are readily distinguished from each other.

Alternatively, the kit is designed for allele specific oligonucleotide ligation. In this embodiment, in addition to the at least one pair of amplification primers, the kit may further contain a pair of detection probes that hybridize to immediately adjacent segments of sequence in one of the strands of the target polynucleotide containing the SNP. One of the two probes is SNP-allele specific; it has a SNP allele-specific nucleotide at either its 5′ or 3′ end. The second probe hybridizes immediately adjacent to the first probe, but is not allele specific. In one variation, the allele-specific probe is fluorescently labeled and the second probe is biotinylated, such that if the two probes are ligated, the resultant ligation product is both fluorescently labeled and biotinylated. Optionally, a third probe may be provided which is specific for the other allele of the SNP. If the optional third probe is provided, its fluorescent label may be distinguishably different from the label on the first probe.

In yet another variation, the kit is designed for a 5′ nuclease assay. In this variation, in addition to the at least one pair of amplification primers, the kit may further contain at least one SNP allele-specific probe which is fluorescently labeled. The allele-specific probe may hybridize to either strand of the amplified double-stranded target polynucleotide. In a preferred embodiment, the allele-specific probe evenly straddles the SNP. That is, the SNP position is approximately in the middle of the allele-specific probe. Optionally, the kit also contains a second allele-specific probe which is specific for another allele of the SNP for which the first probe is specific. The fluorescent label on the optional second probe may be distinguishably different from the label on the first probe.

Any of the above kit variations may contain sets of primers and probes for more than one SNP position. For instance, the SNPs detected may be any combination of the SNP described herein and other SNPs. Probes and/or primers for other SNPs diagnostic for a particular disease or malady may also be included. Any kit may optionally contain one or more nucleic acids that serve as a positive control for the amplification primers and/or the probes. Any kit may optionally contain an instruction material for performing diagnosis, particularly the interpretation of results as they relate to the prediction of IOP reduction following treatment with a topical beta-blocker.

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1

### Subject Selection and General Methods for Genotype Analysis

The electronic medical records of adults enrolled in the population-based Marshfield Clinic Personalized Medicine Research Project (PMRP) were searched from 1960 to 2005 to identify subjects with a diagnosis of ocular hypertension or glaucoma. PMRP is a population-based biobank with stored DNA and serum samples for more than 19,000 subjects aged 18 years and older. All participants gave written, informed consent for the project, which includes access to Marshfield Clinic medical records for phenotyping. More than 95% of the residents within the geographic area selected for PMRP use Marshfield Clinic for their health care, thus allowing for population-based epidemiologic research within the Marshfield Clinic system. Marshfield Clinic is an integrated regional health care system with 700 physicians in 41 locations serving approximately 360,000 patients throughout central and northern Wisconsin. All major medical specialties and subspecialties, except whole organ transplant, are covered within the Clinic system. Except for the city of Marshfield, Marshfield Epidemiologic Study Area (MESA) residents reside rurally or in small towns or villages. The overall project, as well as this sub-study, was approved by the Marshfield Clinic Institutional Review Board.

The medical records were manually abstracted for all IOP measurements, glaucoma diagnoses and surgeries for glaucoma. The medical records were also manually abstracted for the concomitant use of systemic medications known to interact with topical beta-blockers (i.e., systemic beta-blockers or selective serotonin reuptake inhibitors [SSRI]). Glaucoma diagnosis was confirmed with medical record evidence of two or more of the following: 1) glaucomatous visual field defect, 2) elevated IOP (>21 mmHg), 3) optic disc cupping (cup/disc ratio ≧0.8), or 4) rim narrowing characteristic of glaucoma. For this study, glaucoma suspects had only one of the preceding characteristics. Ten percent of the charts were abstracted twice for quality assurance purposes. The IOP prior to topical beta-blocker prescription and the lowest IOP in the first 3 months after prescription of a topical beta-blocker were used to classify case or control status. Subjects were classified as cases for this study if their IOP in the first 3 months after being prescribed a topical beta-blocker decreased by <20%. Subjects with IOPs that decreased 20% or greater were classified as controls.

Stored DNA samples were genotyped from the subjects who had used topical beta-blockers and had baseline and follow-up IOP measurements within the first 3 months. TaqMan™ assays were purchased from Applied Biosystems (ABI), Inc. (Foster City, Calif.). Validated assays were purchased for CYP2D6 Cys188Thr, Gly1934Ala, and Cys2838Thr. Custom assays were designed by ABI for the other three CYP2D6 polymorphisms Gly17949Cys, Ala2637 deleted and G4268C necessary to assign a common haplotype. CYP2D6 genotypes were categorized functionally as extensive metabolizers (*1*1, *1*2, *2*2), intermediate metabolizers (*1*10, *1*3, *1*4, *2*10, *2*3, *2*4) and poor metabolizers (*3*4, *4*4). Assignment of metabolizer status was made on the following basis: extensive metabolizer (EM): 2 normal alleles (*1 and *2 are normal); intermediate metabolizer (IM): 1 abnormal allele; and poor metabolizer (PM): 2 abnormal alleles.

Pre-made assays were purchased from ABI for the beta-1 adrenergic receptor genes, ADRB1 Ser49Gly and Arg389Gly. Custom-made assays were purchased from ABI for the beta-2 adrenergic receptor (ADRB2 Gly16Arg and Gln27Glu) and beta-3 adrenergic receptor (ADRB3 Trp64Arg). Combinations of minor alleles for genotypes in the ADRB1, 2 and 3 SNPs were also calculated as suggested in a review of the pharmacogenetics of human beta-adrenergic receptors. For optineurin Glu50Lys and Met98Lys, assays were purchased from ABI. Custom assays were developed for myocillin Gln368Stop and −1000 Gly/Cys. Myocillin and optineurin were included because of their known genetic risk in certain forms of open-angle glaucoma. Test assays were set up, and if there was a clear distinction between 11, 12 and 22 alleles, then the assay was run on all samples. The allele frequencies were compared to the known allele frequencies in dbSNP and were consistent with the known frequencies.

Data was entered twice and verified. The statistical package available under the federal trademark SPSS® Version 15.0 (SPSS, Chicago, Ill.) was used for the statistical analyses. Chi-square analysis and Fisher's exact test were used to compare proportions. Logistic regression was used to identify independent predictors of IOP response. Ninety-five percent confidence intervals (CI) were calculated using the exact binomial distribution. A P-value <0.05 was considered statistically significant.

### Example 2

### Glaucoma and Intraocular Pressure in Study Subjects

As of Dec. 31, 2005, 18,773 adults were enrolled in PMRP; all were included in this study. The overall rate of definite glaucoma in subjects aged 50 years and older was 2.07% (95% CI=1.20-2.38) and the rate of treated ocular hypertension was 1.42% (95% CI=1.19-1.69). Topical beta-blockers were prescribed for 343 eyes of 215 PMRP subjects. Of these, 5 subjects had SSRI medications at the time their topical beta-blockers were excluded from the study. Hence a total of 210 subjects available for genotyping were included in this study. The gender distribution was 58.1% female (n=122) and 41.9% male (n=88). Their mean age on Dec. 31, 2005 was 63.8 years (SD=11.3), ranging from 33.8 to 85.4 years. Forty-three percent (n=90) reported a family history of glaucoma on the initial questionnaire administered at the time of enrollment into PMRP. Fifteen (7.1%) of the group were taking systemic beta-blockers at the time of their topical beta-blockers. All polymorphisms were found to be in Hardy Weinberg equilibrium. The frequency of alleles defining CYP2D6 haplotype were as follows: 40.1%*1, 35.2%*2, 3.3%*3, 19.5%*4, and 1.9%*10, which is similar to what has been reported previously in Caucasians. Of the 210 subjects genotyped, 28 (13.3%) had allele combinations that could not be assigned to common CYP2D6 haplotypes.

The mean IOP in the right eye at baseline and follow-up was 24.9 (SD=5.9) and 19.1 (SD=3.9), respectively. The mean IOP in the left eye at baseline and follow-up was 24.8 (SD=5.9) and 18.8 (SD=3.6), respectively. The greatest change in IOP in either eye in the first 3 months ranged from −70.8 mm Hg to +25.0 mm Hg (median=−23.3). A reduction of IOP of 20% or greater in the first 3 months was observed for 55.2% (n=91) of right eyes, 54.4% (n=92) of left eyes. A ≧20% reduction in IOP in the first 3 months in one or both eyes was observed in 61.0% (n=128) of the subjects. Subjects treated with beta-blockers alone had significantly higher response rates than subjects treated with a combination of beta-blockers and other IOP lowering medications (70.3% versus 40.1%, chi-squared=14.7, P-value=43.1). In this drug-exposed study cohort (n=210), age was not related to IOP response (t-test=1.48, P-value=0.14), nor was use of systemic beta-blockers (chi-squared=0.01, P-value=0.94). Males were significantly more likely than females to have either one or both eyes respond with a ≧20% drop in IOP with topical beta-blockers (69.3% versus 54.9%, chi-squared=4.5, P=0.04).

### Example 3

### Genotype Distribution for CYP2D6, ADRB and Glaucoma Disease Genes

Tables 1-3 display the comparison of genotype distribution for CYP2D6, ADRB and glaucoma disease genes, respectively, between subjects who did or did not have a ≧20% drop in their IOP. Variables were created to combine genotypes. Predicted CYP2D6 phenotypes were not related to IOP response (Table 1), nor were optineurin or myocillin gene polymorphisms (Table 3). Optineurin E50K was not polymorphic in this population. The ADRB coding SNPs were not associated with IOP lowering efficacy in univariate analyses (Table 2). Variables that combined the minor alleles for the five ADRB SNPs were created. None of them was found to be statistically significant (data not presented).

TABLE 1
Unadjusted comparison of CYP2D6 functional genotype between subjects
who did and did not have a ≧20% drop in their IOP in either
eye in the first 3 months of topical beta-blocker use

| | Both eyes did | Either or both | |
|---|---|---|---|
| | not have 20% | eyes had 20% | |
| CYP2D6 activity | drop in IOP | drop in IOP | Chi-squared |
| (based on haplotype) | (n = 74) | (n = 108) | P-value |
| Extensive metabolizer | 37, 50.0% | 65, 60.2% | |
| Intermediate metabolizer | 31, 41.9% | 39, 36.1% | |
| Poor metabolizer | 6, 8.1% | 4, 3.7% | 2.74, 0.25 |

IOP, intraocular pressure.

TABLE 2
Unadjusted comparison of ADRB genotypes between subjects
who did and did not have a ≧20% drop in their IOP in either
eye in the first 3 months of topical beta-blocker use

| | Both eyes did | Either or | | |
|---|---|---|---|---|
| | not have | both eyes had | | Sequence |
| ADRB | 20% drop in | 20% drop in | Chi-squared, | Identification |
| Genotype | IOP (n = 82) | IOP (n = 128) | P-value | Numbers |
| rs1801252, | | | | SEQ. ID |
| ADRB1, | | | | NO. 3 (DNA) |
| Ser49Gly | | | | SEQ. ID |
| | | | | NO. 4 (protein) |
| AA | 63, 76.8% | 103, 80.5% | | |
| AG | 17, 20.7% | 25, 19.5% | | |
| GG | 2, 2.4% | 0, 0% | 3.24, 0.20 | |
| rs1801253, | | | | SEQ. ID |
| ADRB1, | | | | NO. 3 (DNA) |
| Arg389Gly | | | | SEQ. ID |
| | | | | NO. 4 (protein) |
| CC | 47, 57.3% | 69, 53.9% | | |
| CG | 33, 40.2% | 50, 39.1% | | |
| GG | 2, 2.4% | 9, 7.0% | 2.14, 0.34 | |
| rs1042713, | | | | SEQ. ID |
| ADRB2, | | | | NO. 1 (DNA) |
| Gly16Arg | | | | SEQ. ID |
| | | | | NO. 2 (protein) |
| AA | 9, 11.0% | 20, 15.8% | | |
| AG | 43, 52.4% | 55, 43.3% | | |
| GG | 30, 36.6% | 52, 40.9% | 1.95, 0.27 | |
| rs1042714, | | | | SEQ. ID |
| ADRB2, | | | | NO. 1 (DNA) |
| Gln27Glu | | | | SEQ. ID |
| | | | | NO. 2 (protein) |
| CC | 22, 26.8% | 46, 35.9% | | |
| CG | 45, 54.9% | 54, 42.2% | | |
| GG | 15, 18.3% | 28, 21.9% | 3.30, 0.19 | |
| rs4994, | | | | SEQ. ID |
| ADRB3, | | | | NO. 5 (DNA) |
| Trp64Arg | | | | SEQ. ID |
| | | | | NO. 6 (protein) |
| CC | 0, 0% | 1, 0.8% | | |
| CT | 11, 13.8% | 18, 14.3% | | |
| TT | 69, 86.3% | 107, 84.9% | 0.65, 0.72 | |

IOP, intraocular pressure.

TABLE 3
Unadjusted comparison of putative glaucoma disease genotypes between
subjects who did and did not have a ≧20% drop in their IOP in
either eye in the first 3 months of topical beta-blocker use

| | Both eyes did | Either or | | |
|---|---|---|---|---|
| | not have | both eyes had | | Sequence |
| Disease | 20% drop in | 20% drop in | Chi-squared, | Identification |
| Genotype | IOP (n = 82) | IOP (n = 127) | P-value | Number |
| rs11258194, | | | | SEQ. ID |
| optineurin | | | | NO. 7 (DNA) |
| | | | | SEQ. ID |
| | | | | NO. 8 (protein) |
| AA | 0, 0% | 1, 0.8% | | |
| AT | 6, 7.3% | 12, 9.4% | | |
| TT | 76, 92.7% | 114, 89.8% | 0.96, 0.62 | |
| Myocillin | | | | SEQ. ID |
| Gly1000Cys | | | | NO. 9 (DNA) |
| | | | | SEQ. ID |
| | | | | NO. 10 (protein) |
| CC | 71, 86.6% | 109, 85.8% | | |
| CG | 11, 13.4% | 18, 14.2% | 0.02, 0.88 | |
| Myocillin | | | | SEQ. ID |
| Gln368Stop | | | | NO. 9 (DNA) |
| | | | | SEQ. ID |
| | | | | NO. 10 (protein) |
| CC | 81, 98.8% | 126, 99.2% | | |
| CT | 0, 0% | 1, 0.8% | 1.00* | |

*Fishers' exact test.
IOP, intraocular pressure.

Table 4 presents the results of the logistic regression models for ADRB2, developed to predict a ≧20% drop in IOP with beta-blocker use, and adjusted for gender, family history of glaucoma and use of systemic beta-blockers. For both ADRB1 and ADRB3, there were too few subjects homozygous for the minor allele to allow for multivariate logistic regression analyses. After adjusting for gender, family history of glaucoma and use of systemic beta-blockers, subjects with the homozygous major allele (CC) genotype for rs1042714 were significantly more likely than subjects with the heterozygous (CG) genotype to experience a ≧20% decrease in their IOP (OR=2.0, 95% CI=1.00-4.02).

TABLE 4
Logistic regression models, adjusted for gender, family history of
glaucoma and use of systemic beta-blockers, to predict ≧20% drop
in IOP with topical beta-blockers for the individual ADRB2 SNPs

| | Odds | 95% confidence | |
|---|---|---|---|
| ADRB2 polymorphism | ratio | interval | P-value |
| rs1042713, (Gly16Arg) | | | |
| AG | 1.00 | | |
| AA | 1.93 | 0.75, 5.01 | 0.36 |
| GG | 1.72 | 0.88, 3.37 | 0.17 |
| rs1042714, (Gln27Glu) | | | |
| CG | 1.00 | | |
| CC | 2.00 | 1.00, 4.02 | 0.05 |
| GG | 1.63 | 0.71, 3.73 | 0.25 |

IOP, intraocular pressure; SNP, single nucleotide polymorphism.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments. All publications, patents and patent applications cited herein are hereby incorporated by reference in their entirety for all purposes.

### REFERENCES

1. Weinreb R N, Khaw P T., Lancet. 2004; 363(9422):1711-1720.
2. Friedman D S et al., Arch Ophthalmol. 2004; 122(4):532-538.
3. Wensor M D et al., Ophthalmology. 1998; 105(4):733-739.
4. Khaw P T, Shah P, Elkington A R. BMJ. 2004; 328(7432):156-158.
5. Maier et al., BMJ. 2005; 331(7509):134.
6. Gurwitz J h et al., Am J Public Health. 1993; 83(5):711-716.
7. Patel S C, Spaeth G L., Ophthalmic Surg. 1995; 26(3):233-236.
8. Owen C G et al., 1994 to 2003. Br J Ophthalmol. 2006; 90(7):861-868.
9. Knox F A et al., Br J Ophthalmol. 2006; 90(2):162-165.
10. McLaren N C, Moroi S E, Pharmacogenomics J. 2003; 3(4):197-201.
11. McGourty J C et al., Clin Pharmacol Ther. 1985; 38(4):409-413.
12. Coleman A L et al., Arch Ophthalmol. 1990; 108(9):1260-1263.
13. Huupponen et al., J Ocul Pharmacol. 1991; 7(2):183-187.
14. Edeki T I et al., JAMA. 1995; 274(20):1611-1613.
15. Stewart W C, Garrison P M., Arch Intern Med. 1998; 158(3):221-226.
16. Jin Y et al., J Natl Cancer Inst. 2005; 97(1):30-39.
17. Nieminen T et al., Eur J Clin Pharmacol. 2005; 61(11):811-819.
18. Johnson J A et al., Clin Pharmacol Ther. 2003; 74(1):44-52.
19. Schwartz S G et al., Ophthalmology. 2005; 112(12):2131-2136.
20. Lanfear D E et al., JAMA. 2005; 294(12):1526-1533.
21. McCarty C A et al., J Glaucoma. 2007 (In Press).
22. McCarty C A et al., Personalized Medicine. 2005; 2(1):49-79.
23. DeStefano F et al., J Clin Epidemiol. 1996; 49(6):643-652.
24. Sachse C et al., Am J Hum Genet. 1997; 60(2):284-295.
25. Bradford L D, Pharmacogenomics. 2002; 3(2):229-243.
26. Taylor M R, Pharmacogenomics J. 2007; 7(1):29-37.
27. Green S A et al., J Biol Chem. 1993; 268(31):23116-23121.
28. Green S A et al., Biochemistry. 1994; 33(32):9414-9419.
29. Green S A et al., Am J Respir Cell Mol Biol. 1995; 13(1):25-33.
30. Mason D A et al., J Biol Chem. 1999; 274(18):12670-12674.
31. Levin M C et al., J Biol Chem. 2002; 277(34):30429-30435.
32. Dishy V et al., N Engl J Med. 2001; 345(14):1030-1035.
33. Drysdale C M et al., Proc Natl Acad Sci USA. 2000; 97(19):10483-10488.
34. Xie H G et al., Pharmacogenetics. 1999; 9(4):511-516.
35. Fuchsjager-Mayrl Get al., Mol Vis. 2005; 11:811-815.
36. McLaren N et al., Arch Ophthalmol. 2007; 125(1):105-111.
37. Inagaki Y et al., Mol Vis. 2006; 12:673-680.
38. Brodde O E, Leineweber K, Pharmacogenet Genomics. 2005; 15(5):267-275.

What is claimed is:

1. A method for identifying a glaucoma-afflicted human who will experience a reduction in intraocular pressure upon treatment with a topical beta-blocker, the method comprising detecting a CC genotype at coding single nucleotide polymorphism (SNP) rs1042714 in the ADRB2 gene (Gln27Glu) in a nucleic acid sample from said human, wherein the presence of the CC genotype indicates that said human will experience a clinically meaningful reduction in intraocular pressure upon treatment with a topical beta-blocker as compared to a human lacking the CC genotype and undergoing the same said treatment.

2. The method according to claim 1, wherein detection is carried out by allele-specific probe hybridization, allele-specific primer extension, allele-specific amplification, sequencing, 5′ nuclease digestion, molecular beacon assay, oligonucleotide ligation assay, restriction fragment size analysis, invasive cleavage assay, branch migration assay, denaturing gradient gel electrophoresis, immunoassay, or single-stranded conformation polymorphism analysis.

3. The method according to claim 1, wherein said human is treated with the topical beta-blocker timolol, levobunolol, betaxolol, metipranolol, carteolol, timolol combined with dorzolamide, or timolol combined with brimonidine.

4. A method of genotype-based glaucoma management to reduce intraocular pressure in a glaucoma-afflicted human, comprising steps of:
(a) detecting a CC genotype at coding single nucleotide polymorphism (SNP) rs1042714 in the ADRB2 gene (Gln27Glu) in a nucleic acid sample from said human, wherein the presence of the CC genotype indicates that said glaucoma-afflicted human will experience a clinically meaningful reduction in intraocular pressure upon treatment with a topical beta-blocker as compared to a glaucoma-afflicted human lacking the CC genotype and undergoing the same said treatment; and
(b) administering topical beta-blocker to the glaucoma-afflicted human in which was detected said CC genotype at coding single nucleotide polymorphism (SNP) rs1042714 in the ADRB2 gene (Gln27Glu) to thereby provide clinically meaningful reduction in intraocular pressure in the glaucoma-afflicted human.

5. The method according to claim 4 in which detection is carried out by allele-specific probe hybridization, allele-specific primer extension, allele-specific amplification, sequencing, 5′ nuclease digestion, molecular beacon assay, oligonucleotide ligation assay, restriction fragment size analysis, invasive cleavage assay, branch migration assay, denaturing gradient gel electrophoresis, immunoassay, or single-stranded conformation polymorphism analysis.

6. The method according to claim 4, wherein said human is treated with the topical beta-blocker timolol, levobunolol, betaxolol, metipranolol, carteolol, timolol combined with dorzolamide, or timolol combined with brimonidine.


SEQUENCE LISTING (10 sequences)

SEQ ID NO: 1 - DNA, 2033 bp, Homo sapiens
gcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatgcc

SEQ ID NO: 2 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 3 - DNA, 2862 bp, Homo sapiens
gcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaagaccgatagcaggtgaactcgaagcccacaatcctcgtctgaatcatccgaggcaaagagaaaagccacggaccgttgcacaaaaaggaaagtttgggaagggatgggagagtggcttgctgatgttccttgttgttttttttttcttttcttttctttcttcttcttttttttttttttttttttttctgtttgtggtccggccttcttttgtgtgtgcgtgtgatgcatctttagatttttttcccccaccaggtggtttttgacactctctgagaggaccggagtggaagatgggtgggttaggggaagggagaagcattaggaggggattaaaatcgatcatcgtggctcccatccctttcccgggaacaggaacacactaccagccagagagaggagaatgacagtttgtcaagacatatttccttttgctttccagagaaatttcattttaatttctaagtaatgatttctgctgttatgaaagcaaagagaaaggatggaggcaaaataaaaaaaaatcacgtttcaagaaatgttaagctcttcttggaacaagccccaccttgctttccttgtgtagggcaaacccgctgtcccccgcgcgcctgggtggtcaggctgagggatttctacctcacactgtgcatttgcacagcagatagaaagacttgtttatattaaacagcttatttatgtatcaatattagttggaaggaccaggcgcagagcctctctctgtgacatgtgactctgtcaattgaagacaggacattaaaagagagcgagagagagaaacagttcagattactgcacatgtggataaaaacaaaaacaaaaaaaaggagtggttcaaaatgccatttttgcacagtgttaggaattacaaaatccacagaagatgttacttgcacaaaaagaaattaaatattttttaaagggagaggggctgggcagatcttaaataaaattcaaactctacttctgttgtctagtatgttattgagctaatgattcattgggaaaatacctttttatactcctttatcatggtactgtaactgtatccatattataaatataattatcttaaggattttttatttttttttatgtccaagtgcccacgtgaatttgctggtgaaagttagcacttgtgtgtaaattctacttcctcttgtgtgttttaccaagtatttatactctggtgcaactaactactgtgtgaggaattggtccatgtgcaataaataccaatgaagcacaatcaa

SEQ ID NO: 4 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 5 - DNA, 2660 bp, Homo sapiens
gctactcctcccccaagagcggtggcaccgagggagttggggtggggggaggctgagcgctctggctgggacagctagagaagatggcccaggctggggaagtcgctctcatgccttgctgtcccctcccctgagccaggtgatttgggagaccccctccttccttctttccctaccgccccacgcgcgacccggggatggctccgtggcctcacgagaacagctctcttgccccatggccggacctccccaccctggcgcccaataccgccaacaccagtgggctgccaggggttccgtgggaggcggccctagccggggccctgctggcgctggcggtgctggccaccgtgggaggcaacctgctggtcatcgtggccatcgcctggactccgagactccagaccatgaccaacgtgttcgtgacttcgctggccgcagccgacctggtgatgggactcctggtggtgccgccggcggccaccttggcgctgactggccactggccgttgggcgccactggctgcgagctgtggacctcggtggacgtgctgtgtgtgaccgccagcatcgaaaccctgtgcgccctggccgtggaccgctacctggctgtgaccaacccgctgcgttacggcgcactggtcaccaagcgctgcgcccggacagctgtggtcctggtgtgggtcgtgtcggccgcggtgtcgtttgcgcccatcatgagccagtggtggcgcgtaggggccgacgccgaggcgcagcgctgccactccaacccgcgctgctgtgccttcgcctccaacatgccctacgtgctgctgtcctcctccgtctccttctaccttcctcttctcgtgatgctcttcgtctacgcgcgggttttcgtggtggctacgcgccagctgcgcttgctgcgcggggagctgggccgctttccgcccgaggagtctccgccggcgccgtcgcgctctctggccccggccccggtggggacgtgcgctccgcccgaaggggtgcccgcctgcggccggcggcccgcgcgcctcctgcctctccgggaacaccgggccctgtgcaccttgggtctcatcatgggcaccttcactctctgctggttgcccttctttctggccaacgtgctgcgcgccctggggggcccctctctagtcccgggcccggctttccttgccctgaactggctaggttatgccaattctgccttcaacccgctcatctactgccgcagcccggactttcgcagcgccttccgccgtcttctgtgccgctgcggccgtcgcctgcctccggagccctgcgccgccgcccgcccggccctcttcccctcgggcgttcctgcggcccggagcagcccagcgcagcccaggctttgccaacggctcgacggggcttcttggggagtttcttaggcctgaaggacaagaagcaacaactctgttgatcagaacctgtggaaaacctctggcctctgttcagaatgagtcccatgggattccccggctgtgacactctaccctccagaacctgacgactgggccatgtgacccaaggagggatccttaccaagtgggttttcaccatcctcttgctctctgtctgagagatgttttctaaaccccagccttgaacttcactcctccctcagtggtagtgtccaggtgccgtggagcagcaggctggctttggtaggggcacccatcacccggcttgcctgtgcagtcagtgagtgcttagggcaaagagagctcccctggttccattccttctgccacccaaaccctgatgagaccttagtgttctccaggctctgtggcccaggctgagagcagcagggtagaaaagaccaagatttggggttttatctctggttcccttattactgctctcaagcagtggcctctctcactttagccatggaatggctccgatctacctcacagcagtgtcagaaggacttcgccagggttttgggagctccagggttcataagaaggtgaaccattagaacagatcccttcttttccttttgcaatcagataaataaatatcactgaatgcagttcatcctcggccccctttccctccgtttgttttcttttcataatccacttactcccttcccttctactctgcgctggcttttgacagaggcagtaaattaggcctaatcctcactcttttcttcctaatcttcatcaaacaaaaaatgaaaagtctgtctggacgaaggggagtgagcttgagcctttgatatcttgctcccccacccttcctgaaactcttgaaatccagttgccattgagtagcaaagccacgctccccacaggacttggacagagggcccacagggggatgggctggctgtggccaggtttagggcagggggcatttgtcccctccatgctataatccagtggtgccttacatggtgtgtgtgtgtgtgtgtgcgtgtgtgtgtgtgtgtgtgtgtctggaggcacaggcacaaagcattgcttgggttggtcaaatgtcttgtgtcataaatatattctgatgtttcccagcctttccacaacctctaccttcccactcaccttccccagctacaaaaatctgtattatcctcttaaagtaaaactggagttacaaaaaaaaaaaaaaaa

SEQ ID NO: 6 - protein, 408 aa, Homo sapiens
MAPWPHENSSLAPWPDLPTLAPNTANTSGLPGVPWEAALAGALLALAVLATVGGNLLVIVAIAWTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCGRRLPPEPCAAARPALFPSGVPAARSSPAQPRLCQRLDGASWGVS

SEQ ID NO: 7 - DNA, 2514 bp, Homo sapiens
ccacgcgtccggccccctccgccaccgccgccgcccgccggcaggttccctggtcagcgtcccatcccggtcgggagttctctccaggcggcacgatgccgaggaaacagtgaccctgagcgaagccaagccgggcggcaggtgtggctttgatagctggtggtgccacttcctggccttggatgagccgtacgcctctgtaaacccaacttcctcacctttgaaacagctgcctggttcagcattaatgaagattagtcagtgacaggcctggtgtgctgagtccgcacatagaagaatcaaaaatgtccaaaatgtaactggagagaaagtgggcaacttttggagaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacgagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcgaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggtaaatgtcagattttttcctccaagagttgtgcttttgtgttatttgttttcactcaaatattttgcctcattattcttgttttaaaagaaagaaaacaggccgggcacagtggctcatgcctgtaatcccagcactttgggaggtcgaggtgggtggatcacttggggtcagggtttgagaccagcctggccaacatggcggaaccctgtctctaccaaaattacaaaaattagccgagcatggtggcgcatgcctgtagtcgcagctactcgcgaggttgaggcaggagaattgcttgaacccaggaagtggcagttgcagtgagccgagacgacaccactgcactccagcctgggtgacagagggagactctgtctcgaaagaaagaaagaaaaaaaaaaaaaaa

SEQ ID NO: 8 - protein, 571 aa, Homo sapiens
MSHQPLSCLTEKEDSPSESTGNGPPHLAHPNLDTFTPEELLQQMKELLTENHQLKEAMKLNNQAMKGRFEELSAWTEKQKEERQFFEIQSKEAKERLMALSHENEKLKEELGKLKGKSERSSEDPTDDSRLPRAEAEQEKDQLRTQVVRLQAEKADLLGIVSELQLKLNSSGSSEDSFVEIRMAEGEAEGSVKEIKHSPGPTRTVSTGTSRSADGAKNYFEHEELTVSQLLLCLREGNQKVERLEVALKEAKERVSDFEKKTSNRSEIETQTEGSTEKENDEEKGPETVGSEVEALNLQVTSLFKELQEAHTKLSEAELMKKRLQEKCQALERKNSAIPSELNEKQELVYTNKKLELQVESMLSEIKMEQAKTEDEKSKLTVLQMTHNKLLQEHNNALKTIEELTRKESEKVDRAVLKELSEKLELAEKALASKQLQMDEMKQTIAKQEEDLETMTILRAQMEVYCSDFHAERAAREKIHEEKEQLALQLAVLLKENDAFEDGGRQSLMEMQSRHGARTSDSDQQAYLVQRGAEDRDWRQQRNIPIHSCPKCGEVLPDIDTLQIHVMDCII

SEQ ID NO: 9 - DNA, 2061 bp, Homo sapiens
gaagcctcaccaagcctctgcaatgaggttcttctgtgcacgttgctgcagctttgggcctgagatgccagctgtccagctgctgcttctggcctgcctggtgtgggatgtgggggccaggacagctcagctcaggaaggccaatgaccagagtggccgatgccagtataccttcagtgtggccagtcccaatgaatccagctgcccagagcagagccaggccatgtcagtcatccataacttacagagagacagcagcacccaacgcttagacctggaggccaccaaagctcgactcagctccctggagagcctcctccaccaattgaccttggaccaggctgccaggccccaggagacccaggaggggctgcagagggagctgggcaccctgaggcgggagcgggaccagctggaaacccaaaccagagagttggagactgcctacagcaacctcctccgagacaagtcagttctggaggaagagaagaagcgactaaggcaagaaaatgagaatctggccaggaggttggaaagcagcagccaggaggtagcaaggctgagaaggggccagtgtccccagacccgagacactgctcgggctgtgccaccaggctccagagaagtttctacgtggaatttggacactttggccttccaggaactgaagtccgagctaactgaagttcctgcttcccgaattttgaaggagagcccatctggctatctcaggagtggagagggagacaccggatgtggagaactagtttgggtaggagagcctctcacgctgagaacagcagaaacaattactggcaagtatggtgtgtggatgcgagaccccaagcccacctacccctacacccaggagaccacgtggagaatcgacacagttggcacggatgtccgccaggtttttgagtatgacctcatcagccagtttatgcagggctacccttctaaggttcacatactgcctaggccactggaaagcacgggtgctgtggtgtactcggggagcctctatttccagggcgctgagtccagaactgtcataagatatgagctgaataccgagacagtgaaggctgagaaggaaatccctggagctggctaccacggacagttcccgtattcttggggtggctacacggacattgacttggctgtggatgaagcaggcctctgggtcatttacagcaccgatgaggccaaaggtgccattgtcctctccaaactgaacccagagaatctggaactcgaacaaacctgggagacaaacatccgtaagcagtcagtcgccaatgccttcatcatctgtggcaccttgtacaccgtcagcagctacacctcagcagatgctaccgtcaactttgcttatgacacaggcacaggtatcagcaagaccctgaccatcccattcaagaaccgctataagtacagcagcatgattgactacaaccccctggagaagaagctctttgcctgggacaacttgaacatggtcacttatgacatcaagctctccaagatgtgaaaagcctccaagctgtacaggcaatggcagaaggagatgctcagggctcctggggggagcaggctgaagggagagccagccagccagggcccaggcagctttgactgctttccaagttttcattaatccagaaggatgaacatggtcaccatctaactattcaggaattgtagtctgagggcgtagacaatttcatataataaatatcctttatcttctgtcagcatttatgggatgtttaatgacatagttcaagttttcttgtgatttggggcaaaagctgtaaggcataatagtttcttcctgaaaaccattgctcttgcatgttacatggttaccacaagccacaataaaaagcataacttctaaaggaagcagaatagctcctctggccagcatcgaatataagtaagatgcatttactacagttggcttctaatgcttcagatagaatacagttgggtctcacataaccctttacattgtgaaataaaattttcttacccaaaaaaaaaaaaaaaaaa

SEQ ID NO: 10 - protein, 504 aa, Homo sapiens
MRFFCARCCSFGPEMPAVQLLLLACLVWDVGARTAQLRKANDQSGRCQYTFSVASPNESSCPEQSQAMSVIHNLQRDSSTQRLDLEATKARLSSLESLLHQLTLDQAARPQETQEGLQRELGTLRRERDQLETQTRELETAYSNLLRDKSVLEEEKKRLRQENENLARRLESSSQEVARLRRGQCPQTRDTARAVPPGSREVSTWNLDTLAFQELKSELTEVPASRILKESPSGYLRSGEGDTGCGELVWVGEPLTLRTAETITGKYGVWMRDPKPTYPYTQETTWRIDTVGTDVRQVFEYDLISQFMQGYPSKVHILPRPLESTGAVVYSGSLYFQGAESRTVIRYELNTETVKAEKEIPGAGYHGQFPYSWGGYTDIDLAVDEAGLWVIYSTDEAKGAIVLSKLNPENLELEQTWETNIRKQSVANAFIICGTLYTVSSYTSADATVNFAYDTGTGISKTLTIPFKNRYKYSSMIDYNPLEKKLFAWDNLNMVTYDIKLSKM